(19) United States
(12) Patent Application Publication
(10) Pub. No.: US 2013/0012751 A1
(43) Pub. Date: Jan. 10, 2013

(54) System and Method For Inhibiting Corrosion

(76) Inventors: Douglas J. Turner, Humble, TX (US); William J. Sisak, Houston, TX (US)

(21) Appl. No.: 13/580,132

(22) PCT Filed: Jan. 6, 2011
(86) PCT No.: PCT/US2011/020385

§ 371 Date: Aug. 20, 2012

Related U.S. Application Data
(60) Provisional application No. 61/313,958, filed on Mar. 15, 2010.

(51) Int. Cl. C07C 7/152

(52) U.S. Cl. 585/868

(57) ABSTRACT

Methods and systems are provided for forming clathrates to reduce or prevent corrosion in hydrocarbon facilities, such as pipelines, among others. An exemplary embodiment provides a method for isolating a corrosive gas in a hydrocarbon stream. The method includes combining a host compound with a hydrocarbon stream comprising a corrosive gas to form a clathrate, wherein a pressure or the reaction, a temperature of the reaction, or both, are controlled to maximize formation of a clathrate of the corrosive gas and minimize the formation of a clathrate of a hydrocarbon in the hydrocarbon stream. The clathrate is separated from the hydrocarbon stream and melted to remove the corrosive gas.

### CROSS-REFERENCE TO RELATED APPLICATION

This application claims the priority benefit of U.S. Provisional Patent Application 61/313,958 filed 15 Mar. 2010 entitled SYSTEM AND METHOD FOR INHIBITING CORROSION, the entirety of which is incorporated by reference herein.

### FIELD

Exemplary embodiments of the present techniques relate to inhibiting corrosion of hydrocarbon systems that contain corrosive gases and water.

### BACKGROUND

Hydrocarbon production is often accompanied by various other constituents, a number of which can be corrosive. In particular, carbon dioxide (CO₂) and hydrogen sulfide (H₂S) are two corrosive gases that may often be found in hydrocarbon reservoirs. These compounds may cause degradation of transportation and processing infrastructure elements, such as pipeline, wells, and the like, which may lead to costly repairs.

As produced, hydrocarbons may contain about 0-8% or more by volume of CO₂ and 0-5% or more, by volume, of H₂S. Further, hydrocarbons may contain varying amounts of water. For example, a hydrocarbon may have 0.1%, 5% or more by volume of water. Corrosion in a hydrocarbon facility may depend on three factors or ingredients, which can be thought of as forming a corrosion triangle. These ingredients are water, a corrosive or electrolytic compound, and a vulnerable metal, such as steel.

FIG. 1 is a diagram of a corrosion triangle 10 illustrating the ingredients of an exemplary corrosion process, i.e., water 12, corrosive gas 14, and a vulnerable metal, such as steel 16. If all of these ingredients are present, corrosion 18 may occur. However, if any ingredient 12, 14, or 16 is limited, corrosion 18 may be reduced or even eliminated. Accordingly, techniques to mitigate corrosion can target spatially separating or removing one or more of the ingredients 12, 14 or 16. For example, dehydration can be used to remove the water 12 or gas separation techniques can be used to remove the corrosive gas 14. Another corrosion prevention technique targets separating the steel 16 from the other ingredients 12 and 14, such as by applying a neutral coating over the steel 16, which essentially removes the steel 16 from the corrosion triangle 10.

As corrosion is an electrochemical process, providing electrons to the metal may lower the corrosion 18. For example, a zinc coating can be applied to the steel 16 to inhibit corrosion 18 by supplying electrons in place of the steel 16. More expensive techniques can replace the steel 16 with a corrosion resistant alloy, such as stainless steel. Further, a zinc, magnesium, or aluminum anodes may be electrically coupled to the steel of a pipeline. The anode provides electrons as it is degraded, protecting the steel. In larger systems, the current flow from a passive sacrificial anode may be insufficient, so a generated current may be used to flow electrons through the pipeline, slowing corrosion.

Each of these methods is useful in certain situations. However, in hydrocarbon production, water and corrosive gases will ultimately need to be separated from the production stream. Accordingly, separation of the corrosive components as early in the production process as possible would be the most useful approach. Water and gas separation methods, such as amine treating, glycol dehydration, and pressure swing adsorption, among others, may be costly and technically challenging to implement in remote applications, such as on the seabed or at a hydrocarbon field.

In addition to increasing corrosion, the presence of water in hydrocarbon streams may cause problems with shipping the hydrocarbon due to the formation of clathrate hydrates with the hydrocarbons. Clathrate hydrates (commonly called hydrates) are weak composites formed from a water matrix and a guest molecule, such as methane, ethane, propane, butane, neopentane, ethylene, propylene, isobutylene, cyclopropane, cyclobutane, cyclopentane, cyclohexane, benzene, carbon dioxide, and hydrogen sulfide, among others. Hydrates may form, for example, at the high pressures and low temperatures that may be found in pipelines and other hydrocarbon processing equipment. After forming, the hydrates can agglomerate, leading to plugging or fouling of the pipeline. Various techniques have been used to lower the probability that clathrates will form or cause plugging or fouling, such as dehydration, thermodynamic inhibition, kinetic inhibition, and anti-agglomerates. As discussed above, dehydration may be difficult to implement in remote production environments.

Thermodynamic hydrate inhibitors, such as methanol, monoethylene glycol, diethylene glycol, triethylene glycol, and potassium formate, among others, lower the formation temperature of the hydrate, which may inhibit the formation of the hydrate under the conditions found in a particular process. However, these materials may be used at high levels (e.g., greater than about 10%) to achieve effective inhibition of hydrate formation.

Kinetic hydrate inhibitors (KHIs), which may also be called low dosage hydrate inhibitors, also slow the formation of hydrates, but not by changing the thermodynamic conditions. Instead, KHIs inhibit the nucleation and growth of the hydrate crystals. Such materials may include, for example, Poly(2-alkyl-2-oxazoline) polymers (or poly(N-acylalkylene imine) polymers), poly(2-alkyl-2-oxazoline) copolymers, and others. See Urdahl, Olav, et al., “Experimental testing and evaluation of a kinetic gas hydrate inhibitor in different fluid systems,” Preprints from the Spring 1997 Meeting of the ACS Division of Fuel Chemistry, 42, 498-502 (American Chemical Society, 1997).

For example, U.S. Pat. No. 6,359,047 discloses a gas hydrate inhibitor. The inhibitor includes, by weight, a copolymer including about 80 to about 95% of polyvinyl caprolactam (VCL) and about 5 to about 20% of N,N-dialkylaminoethyl(meth)acrylate or N-(3-dimethylaminopropyl) methacrylamide. As another example, U.S. Pat. No. 5,874,660 discloses a method for inhibiting hydrate formation. The method be used in treating a petroleum fluid stream such as natural gas conveyed in a pipe to inhibit the formation of a hydrate restriction in the pipe. The hydrate inhibitor used for practicing the method is selected from the family of substantially water soluble copolymers formed from N-methyl-N-vinylacetamide (VIMA) and one of three comonomers, vinylpyrrolidone (VP), vinylpiperidone (VPip), or vinylcaprolactam (VCap). VIMA/VCap is the preferred copolymer. These copolymers may be used alone or in combination with each other or other hydrate inhibitors. Preferably, a solvent, such as water, brine, alcohol, or mixtures thereof, is used to produce an inhibitor solution or mixture to facilitate treatment of the petroleum fluid stream.

Surface active agents (surfactants) may function both as KHIs and as anti-agglomeration agents (anti-agglomerates). Anti-agglomerates may prevent the agglomeration, or self-sticking, of small hydrate crystals into larger hydrate crystals or groups of crystals. For example, U.S. Pat. Nos. 5,841,010 and 6,015,929 disclose the use of surface active agents as gas hydrate inhibitors for inhibiting the formation (nucleation, growth and agglomeration) of clathrate hydrates. The methods comprise adding into a mixture comprising hydrate forming substituents and water, an effective amount of a hydrate inhibitor selected from the group consisting of anionic, cationic, non-ionic and zwitterionic hydrate inhibitors. The hydrate inhibitor has a polar head group and a nonpolar tail group not exceeding 12 carbon atoms in the longest carbon chain. The anti-agglomeration agents may allow for the formation of a flowable slurry, i.e., hydrates that can be carried by a flowing hydrocarbon without sticking to each other.

Related information may be found in U.S. Pat. Nos. 6,957,146; 5,936,040; 5,841,010; and 5,744,665. Further information may be found in: U.S. Patent Application Publication Nos. 2004/0133531, 20060092766, 2008/0312478 and 2007/0129256; Sloan, E. D., “Gas Hydrate Tutorial,” Preprints from the Spring 1997 Meeting of the ACS Division of Fuel Chemistry, 42(2), 449-56 (American Chemical Society, 1997); and in Talley, L. D. and Edwards, M., “First Low Dosage Hydrate Inhibitor is Field Proven in Deepwater,” Pipeline and Gas Journal 44, 226 (1999).

The techniques discussed above may help to prevent the formation of hydrates or the plugging of lines by hydrates, but may not help in slowing or preventing corrosion. Further, the materials used as thermodynamic or kinetic hydrate inhibitors may not be compatible with anti-corrosion agents such as coatings or chemical agents used for corrosion protection. Finally, the injection or use of these materials may not be appropriate in numerous reservoirs or process situations, due to cost or complexity.

Hydrates have been tested to determine whether they can be used to remove CO₂ from H₂ in a synthesis gas stream prior to combustion in a power plant. See Tam, S. S., et al., “A High Pressure Carbon Dioxide Separation Process for IGCC Plants,” Proceedings of the First National Conference on Carbon Sequestration (United States Dept. of Energy, National Energy Technology Laboratory, 2001). It was determined that hydrates could be used to remove CO₂ and H₂S from a synthesis gas stream (for example, generated by a partial oxidation of coal followed by a water gas shift reaction). The primary component in synthesis gas is H₂, which does not form hydrates under normal process conditions (for example, less than about 1000 psia, or greater than about 77 F), which simplifies the formation and separation of other hydrates from the H₂ stream.

### SUMMARY

An exemplary embodiment of the present techniques provides a method for isolating a corrosive gas in a hydrocarbon stream. The method includes reacting a host compound with the hydrocarbon stream comprising the corrosive gas. The pressure, temperature, or both, of the reaction, are controlled to maximize formation of a clathrate of the corrosive gas and minimize formation of a clathrate of a hydrocarbon in the hydrocarbon stream. The clathrate of the corrosive gas is separated from the hydrocarbon stream and melt to remove the corrosive gas.

The method may include placing a reactor configured to form the clathrate of the corrosive gas at a first location in a hydrocarbon transport system; placing a separator configured to remove the clathrate of the corrosive gas at a second location in the hydrocarbon transport system; and placing a melter configured to melt the clathrate of the corrosive gas at a third location in the hydrocarbon transport system.

Exemplary embodiments may include reacting the host compound with the hydrocarbon stream at a wellhead to form the clathrate of the corrosive gas and pumping a slurry comprising the hydrocarbon and the clathrate of the corrosive gas to a destination. A slurry of the clathrate of the corrosive gas may be formed in a reservoir and flowed to a separation system at a surface, such as the surface of the earth or an ocean. A produced sour water, the corrosive gas, or both, may be reinjected into a producing or non-producing reservoir. An anti-agglomerate may be added to the hydrocarbon stream.

Various corrosion prevention techniques may be used in exemplary embodiments of the present techniques, including, for example, adding a corrosion inhibitor to the hydrocarbon stream, using a cathodic protection system, applying a coating to a metal surface in a hydrocarbon transport system, forming a part in the hydrocarbon transport system from a corrosion resistant alloy, or any combinations thereof.

Another exemplary embodiment of the present techniques provides a system for transporting a hydrocarbon through a transportation infrastructure. The system may include a reactor configured to form a clathrate between a host compound and a corrosive gas in a hydrocarbon stream, wherein the reactor comprises a heat exchanger configured to control a temperature of the reactor to minimize a formation of a hydrocarbon clathrate. The system may also include a separator configured to remove the clathrate from the hydrocarbon stream and a melter configured to melt the clathrate and release the corrosive gas.

The system may include a pipeline configured to transport a slurry comprising the clathrate in the hydrocarbon stream. The system may also include a vessel configured to function as the reactor, the separator, and the melter. In an embodiment, the system may include a vessel configured to function as the separator and the melter. The reactor may include a static mixer. A water injection port may be located upstream of the static mixer. An injection system may be configured to inject the corrosive gas released from melting the clathrate into a well. The host compound may include water. The corrosive gas may include carbon dioxide, hydrogen sulfide, or any combination thereof.

Another exemplary embodiment provides a method for producing a hydrocarbon. The method may include producing a hydrocarbon stream that includes a corrosive gas, reacting a host compound with the hydrocarbon stream to form a slurry of a clathrate of the corrosive gas in the hydrocarbon stream, and transporting the slurry to a destination through a pipeline. The clathrate may be separated from the hydrocarbon stream and melted to remove the corrosive gas. The method may include melting the clathrate in the pipeline and separating the corrosive gas from the hydrocarbon stream at the destination.

### DESCRIPTION OF THE DRAWINGS

The advantages of the present techniques are better understood by referring to the following detailed description and the attached drawings, in which:

FIG. 1 is a diagram of a corrosion triangle illustrating the ingredients of a corrosion process, i.e., water, corrosive gas, and a vulnerable metal;

FIG. 2 is a graph of the hydrate equilibrium curves for methane, carbon dioxide, and hydrogen sulfide, in accordance with an exemplary embodiment of the present techniques;

FIG. 3 is a schematic illustrating the effect of forming a hydrate on corrosion, in accordance with an exemplary embodiment of the present techniques;

FIG. 4 is a schematic illustrating the effect of consuming a corrosive gas in the formation of a hydrate, in accordance with an exemplary embodiment of the present techniques;

FIG. 5 is a schematic illustrating the effect of consuming the water in the formation of a hydrate, in accordance with an exemplary embodiment of the present techniques;

FIG. 6 is a block diagram of a system for shipping a hydrate slurry in a hydrocarbon, in accordance with an exemplary embodiment of the present techniques;

FIG. 7 is a block diagram of a separation tower that can use clathrates, such as hydrates, to separate corrosive gases from a hydrocarbon, in accordance with an exemplary embodiment of the present techniques;

FIG. 8 is a block diagram that is useful in explaining the operation of the separation tower of FIG. 7 to purify a hydrocarbon stream, in accordance with an exemplary embodiment of the present techniques;

FIG. 9 is a process flow diagram showing a method for using clathrates to remove corrosive gases from hydrocarbons, in accordance with exemplary embodiments of the present techniques;

FIG. 10 is a bar chart comparing the mole fractions of methane and CO₂ in a feed phase and a hydrate phase, in accordance with exemplary embodiments of the present techniques;

FIG. 11 is a McCabe-Thiele plot for a theoretically staged separation column for the separation of CO₂ from methane, in accordance with an exemplary embodiment of the present techniques;

FIG. 12 is a bar chart comparing the mole fractions of CH₄ and H₂S in a feed phase and a hydrate phase, in accordance with exemplary embodiments of the present techniques; and

FIG. 13 is a McCabe-Thiele plot for a theoretically staged separation column for H₂S separation from methane, in accordance with an exemplary embodiment of the present techniques.

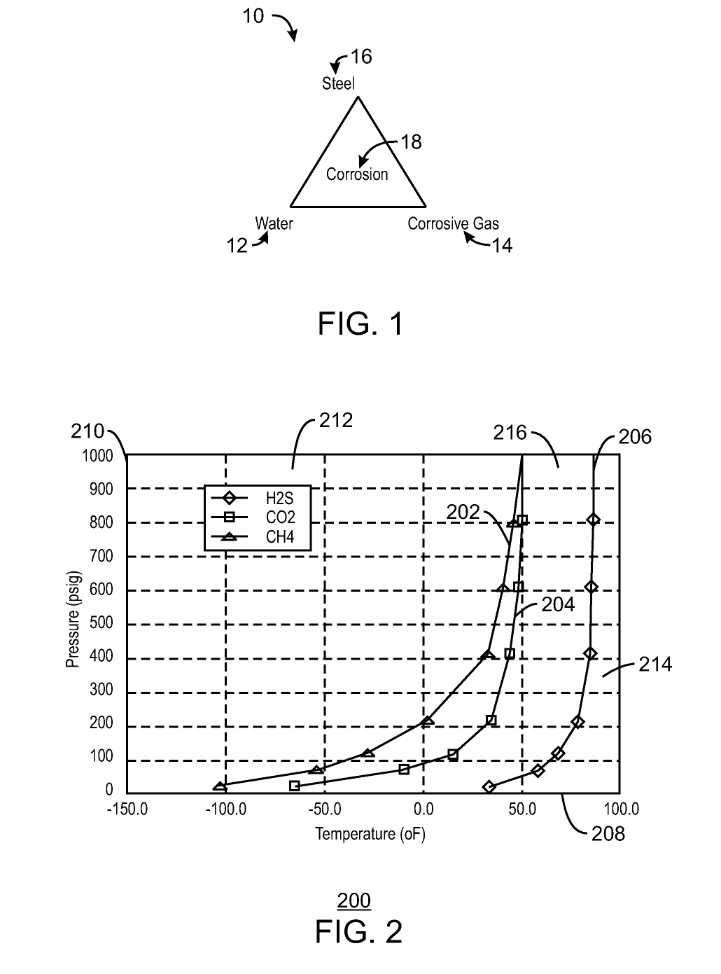

### DETAILED DESCRIPTION

In the following detailed description section, specific embodiments of the present techniques are described. However, to the extent that the following description is specific to a particular embodiment or a particular use of the present techniques, this is intended to be for exemplary purposes only and simply provides a description of the exemplary embodiments. Accordingly, the techniques are not limited to the specific embodiments described below, but rather, include all alternatives, modifications, and equivalents falling within the true spirit and scope of the appended claims.

At the outset, for ease of reference, certain terms used in this application and their meanings as used in this context are set forth. To the extent a term used herein is not defined below, it should be given the broadest definition persons in the pertinent art have given that term as reflected in at least one printed publication or issued patent. Further, the present techniques are not limited by the usage of the terms shown below, as all equivalents, synonyms, new developments, and terms or techniques that serve the same or a similar purpose are considered to be within the scope of the present claims.

As used herein, the terms “acid gas” and “corrosive gas” are used to refer to a gas encountered in “sour” natural gas streams or petroleum reservoirs. The gases most commonly removed from sour gas or liquid streams are carbon dioxide (CO₂) and hydrogen sulfide (H₂S). Other examples of acid gases include carbonyl sulfide, carbon disulfide, mercaptans and other sulfides.

As used herein, “clathrate” is a weak composite made of a host compound that forms a basic framework and a guest compound that is held in the host framework by inter-molecular interaction, such as hydrogen bonding, Van der Waals forces, and the like. Clathrates may also be called host-guest complexes, inclusion compounds, and adducts. As used herein, “clathrate hydrate” and “hydrate” are interchangeable terms used to indicate a clathrate having a basic framework made from water as the host compound. A hydrate is a crystalline solid which looks like ice, and forms when water molecules form a cage-like structure around a “hydrate-forming constituent.”

As used herein, a “hydrate-forming constituent” refers to a compound or molecule in petroleum fluids, including natural gas, that forms hydrate at elevated pressures and/or reduced temperatures. Illustrative hydrate-forming constituents include, but are not limited to, hydrocarbons such as methane, ethane, propane, butane, neopentane, ethylene, propylene, isobutylene, cyclopropane, cyclobutane, cyclopentane, cyclohexane, and benzene, among others. Hydrate-forming constituents can also include non-hydrocarbons, such as oxygen, nitrogen, hydrogen sulfide, carbon dioxide, sulfur dioxide, and chlorine, among others.

As used herein, a “compressor” is a machine that increases the pressure of a gas by the application of work (compression). Accordingly, a low pressure gas (for example, 5 psig) may be compressed into a high-pressure gas (for example, 1000 psig) for transmission through a pipeline, injection into a well, or other processes.

As used herein, a “column” means a distillation or fractionation column or zone, i.e., a contacting column or zone, wherein liquid and vapor phases can be counter-currently contacted to effect separation of compounds in a mixture of phases. For example, a separation in a liquid-vapor system may be performed by contacting of the vapor and liquid phases on a series of vertically spaced trays or plates mounted within the column and/or on packing elements such as structured or random packing. Further, a separation of compounds in a mixture of solid, liquid, and vapor phases may be effected by counter-current flow of the solid and/or liquid phases in an opposite direction to a vapor phase. A double column comprises a higher pressure column having its upper end in heat exchange relation with the lower end of a lower pressure column.

As used herein, a “facility” as used herein is a representation of a tangible piece of physical equipment through which hydrocarbon fluids are either produced from a reservoir or injected into a reservoir. In its broadest sense, the term facility is applied to any equipment that may be present along the flow path between a reservoir and the destination for a hydrocarbon product. Facilities may comprise production wells, injection wells, well tubulars, wellhead equipment, gathering lines, manifolds, pumps, compressors, separators, surface flow lines and delivery outlets. In some instances, the term “surface facility” is used to distinguish those facilities other than wells. A “facility network” is the complete collection of facilities that are present in the model, which would include all wells and the surface facilities between the wellheads and the delivery outlets.

As used herein, a “formation” is any finite subsurface region. The formation may contain one or more hydrocarbon-containing layers, one or more non-hydrocarbon containing layers, an overburden, and/or an underburden of any subsurface geologic formation. An “overburden” and/or an “underburden” is geological material above or below the formation of interest.

As used herein, the term “gas” is used interchangeably with “vapor,” and means a substance or mixture of substances in the gaseous state as distinguished from the liquid or solid state. Likewise, the term “liquid” means a substance or mixture of substances in the liquid state as distinguished from the gas or solid state. As used herein, “fluid” is a generic term that may include either a gas or vapor.

As used herein, “kinetic hydrate inhibitor” refers to a molecule and/or compound or mixture of molecules and/or compounds capable of decreasing the rate of hydrate formation in a petroleum fluid that is either liquid or gas phase. A kinetic hydrate inhibitor can be a solid or liquid at room temperature and/or operating conditions. The hydrate formation rate can be reduced sufficiently by a kinetic hydrate inhibitor such that no hydrates form during the time fluids are resident in a pipeline at temperatures below the hydrate formation temperature.

For the inhibition of hydrate formation by thermodynamic or kinetic hydrate inhibitors, As used herein, the term “minimum effective operating temperature” refers to the temperature above which hydrates do not form in fluids containing hydrate forming constituents during the time the fluids are resident in a pipeline. For thermodynamic inhibition only, the minimum effective operating temperature is equal to the thermodynamically inhibited hydrate formation temperature. For kinetic hydrate inhibitors, the minimum effective operating temperature is lower than the thermodynamically inhibited hydrate formation temperature. For the combination of thermodynamic and kinetic inhibition, the minimum effective operating temperature may be even less than the thermodynamically inhibited hydrate formation temperature by itself.

As used herein, the term “natural gas” refers to a multi-component gas obtained from a crude oil well (associated gas) or from a subterranean gas-bearing formation (non-associated gas). The composition and pressure of natural gas can vary significantly. A typical natural gas stream contains methane (C₁) as a significant component. Raw natural gas will also typically contain ethane (C₂), higher molecular weight hydrocarbons, one or more acid gases (such as carbon dioxide, hydrogen sulfide, carbonyl sulfide, carbon disulfide, and mercaptans), and minor amounts of contaminants such as water, nitrogen, iron sulfide, wax, and crude oil.

As used herein, a “McCabe-Thiele plot” is a graph of an equilibrium concentration between two chemical components showing the concentration ratio of the components in each of two phases. In the graph, operating lines are used to define the mass balance relationships between the components. A McCabe-Thiele plot may be used to design a separation system based on the different concentrations of each of the components in each of the different phases. While McCabe-Thiele plots are generally used to design columns based on vapor-liquid equilibriums, they may be used in any phase equilibrium, such as the clathrate-liquid equilibrium discussed herein.

As used herein, “pressure” is the force exerted per unit area by the gas on the walls of the volume. Pressure can be shown as pounds per square inch (psi). “Atmospheric pressure” refers to the local pressure of the air. “Absolute pressure” (psia) refers to the sum of the atmospheric pressure (14.7 psia at standard conditions) plus the gage pressure (psig). “Gauge pressure” (psig) refers to the pressure measured by a gauge, which indicates only the pressure exceeding the local atmospheric pressure (i.e., a gauge pressure of 0 psig corresponds to an absolute pressure of 14.7 psia). The term “vapor pressure” has the usual thermodynamic meaning. For a pure component in an enclosed system at a given pressure, the component vapor pressure is essentially equal to the total pressure in the system.

As used herein, the terms “produced fluids” and “production fluids” refer to liquids or gases removed from a subsurface formation. Such produced fluids may include liquids, such as oil or water, and gases, such as natural gas, CO₂, and H₂S, among others.

As used herein, “reflux” is defined as a stream introduced into a distillation column at any location above the location at which the feed is introduced into the column, wherein the reflux comprises one or more components previously withdrawn from the column. Reflux typically is liquid but may be a vapor-liquid mixture or a vapor.

As used herein, “sour gas” generally refers to natural gas containing corrosive gases such as hydrogen sulfide (H₂S) and carbon dioxide (CO₂). When the H₂S and CO₂ have been removed (e.g., to less than 5 ppm) from the natural gas feedstream, the gas is classified as “sweet.” As used herein, the term “sour gas” is applied to natural gases that include H₂S because of the odor that is emitted even at low concentrations from an unsweetened gas. Furthermore, H₂S is corrosive to most metals normally associated with gas pipelines so that processing and handling of sour gas may lead to premature failure of such systems.

As used herein, “substantial” when used in reference to a quantity or amount of a material, or a specific characteristic thereof, refers to an amount that is sufficient to provide an effect that the material or characteristic was intended to provide. The exact degree of deviation allowable may in some cases depend on the specific context.

As used herein, “thermodynamic hydrate inhibitor” refers to a molecule and/or compound, or mixture of molecules and/or compounds capable of reducing the hydrate formation temperature in a petroleum fluid that is either liquid or gas phase. For example, the minimum effective operating temperature of a petroleum fluid can be reduced by at least 1.5° C., 3° C., 6° C., 12° C., or 25° C., due to the addition of one or more thermodynamic hydrate inhibitors. Generally the THI is added to a system in au amount sufficient to prevent the formation of any hydrate.

As used herein, the terms “well” or “wellbore” refer to a hole in the subsurface made by drilling or insertion of a conduit into the subsurface. The terms are interchangeable when referring to an opening in the formation. A well may have a substantially circular cross section, or other cross-sectional shapes (for example, circles, ovals, squares, rectangles, triangles, slits, or other regular or irregular shapes). Wells may be cased, cased and cemented, or open-hole well, and may be any type, including, but not limited to a producing well, an experimental well, an exploratory well, or the like. A well may be vertical, horizontal, or any angle between vertical and horizontal (a deviated well), for example a vertical well may comprise a non-vertical component.

### Overview

Exemplary embodiments of the present technique provide systems and methods for forming clathrates, such as hydrates, to reduce corrosion in facilities without having to separate out corrosive gases. The formation of the hydrate has the effect of consuming the water, which may reduce corrosion in its presence. Furthermore, a hydrate formed with hydrogen sulfide (H₂S), carbon dioxide (CO₂), or a mixture of these gases as the guest molecules, consumes these ingredients as well, which may also reduce corrosion.

FIG. 2 is a graph 200 of the hydrate equilibrium curves for methane 202, carbon dioxide 204, and hydrogen sulfide 206, in accordance with an exemplary embodiment of the present techniques. In the graph 200, the x-axis 208 represents the temperature of a system in degrees Fahrenheit, while the y-axis 210 represents the pressure of the system in pounds per square inch, gauge (psig). The equilibrium curves indicate the pressure and temperature point at which the hydrate is in equilibrium with the individual components, for example, water and a particular gas. In a first region 212, generally at higher pressure and lower temperatures, formation of the hydrates of all components, including a hydrocarbon, may occur. In a second region 214, generally at lower pressures and higher temperatures, the decomposition of the hydrates of all components may occur. However, in regions between the curves, such as region 216, formation of one hydrate, such as a hydrate of H₂S 206 may still be occurring, while another hydrate, such as a hydrate of methane 202, may be decomposing.

Thus, as indicated by the graph 200, CO₂ and H₂S form more stable hydrates than other natural gases, such as methane. As a result, these corrosive components can be preferentially selected to form hydrates at a selected temperature and pressure, which may be useful for purifying a natural gas. Further, the removal of the water and corrosive gas may reduce the tendency of the mixture to corrode a pipeline or other facility.

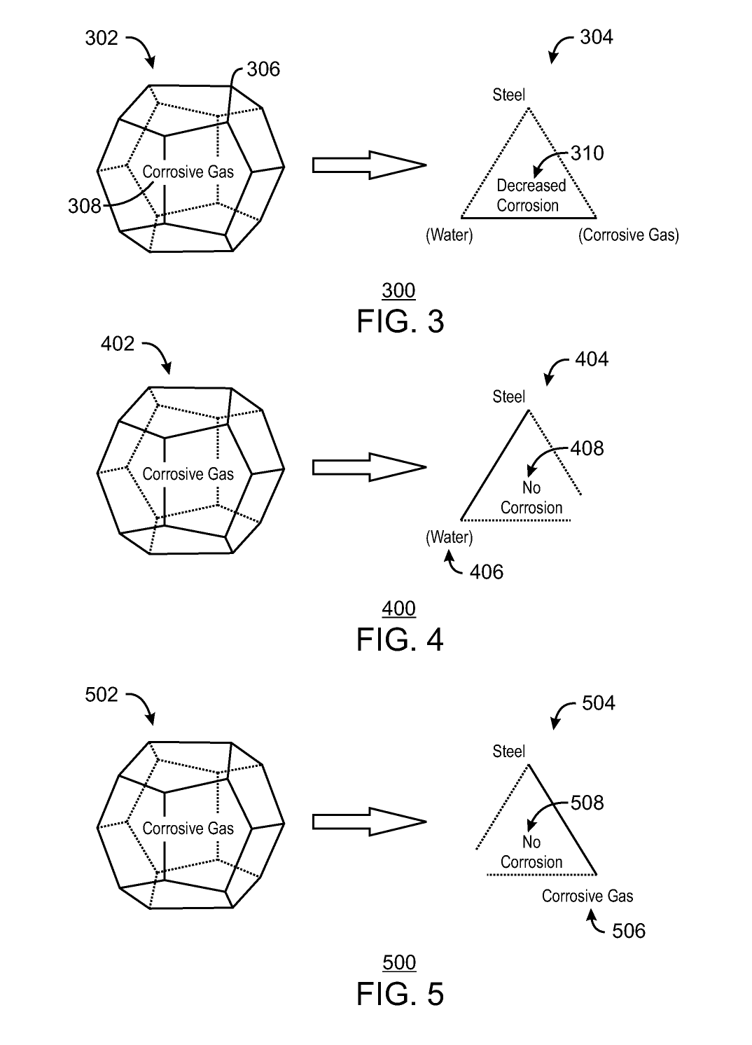

FIG. 3 is a schematic 300 illustrating the effect of forming a hydrate 302 on corrosion, in accordance with an exemplary embodiment of the present techniques. As illustrated by a corrosion triangle 304, as the water matrix 306 forms and traps the corrosive gas 308, the amount of both ingredients may be reduced, decreasing the corrosion 310. In this example, there is incomplete hydrate formation, leaving some water and corrosive gas still available for causing corrosion 310. Such a situation may occur if the pressure and temperature were between the equilibrium curves for H₂S 206 and CO₂ 204 (FIG. 2), thus, allowing a H₂S hydrate to form, but not allowing the formation of a CO₂ hydrate. When hydrate formation occurs at a temperature and pressure favorable for reaction of all corrosive gases, the hydrate formation may continue until the corrosive gases are consumed, or the water is consumed.

FIG. 4 is a schematic 400 illustrating the effect of consuming the corrosive gas in the formation of a hydrate 402, in accordance with an exemplary embodiment of the present techniques. As shown in the diagram 400, upon complete reaction the corrosion triangle 404 may be broken as the corrosive gas is substantially removed. In this case, the water 406 may be substantially reduced in the formation of the hydrate. With the corrosion triangle broken, corrosion 408 may be prevented. Similarly, removal of the water may reduce or eliminate corrosion.

FIG. 5 is a schematic 500 illustrating the effect of consuming the water in the formation of a hydrate 502, in accordance with an exemplary embodiment of the present techniques. As shown in the diagram 500, upon complete reaction, the corrosion triangle 504 may be broken as the water is substantially removed and the corrosive gas 506 is reduced. With the corrosion triangle broken, corrosion 508 may be prevented. The changing gas composition may drive the reaction to a new equilibrium. In this case, neither the water nor the corrosive gas is a limiting reagent. As a new equilibrium is established, both components of water and corrosive gas are reduced. This is a similar situation to that discussed with respect to FIG. 3, which illustrates that with the reduction in corrosion ingredients, corrosion may also be reduced.

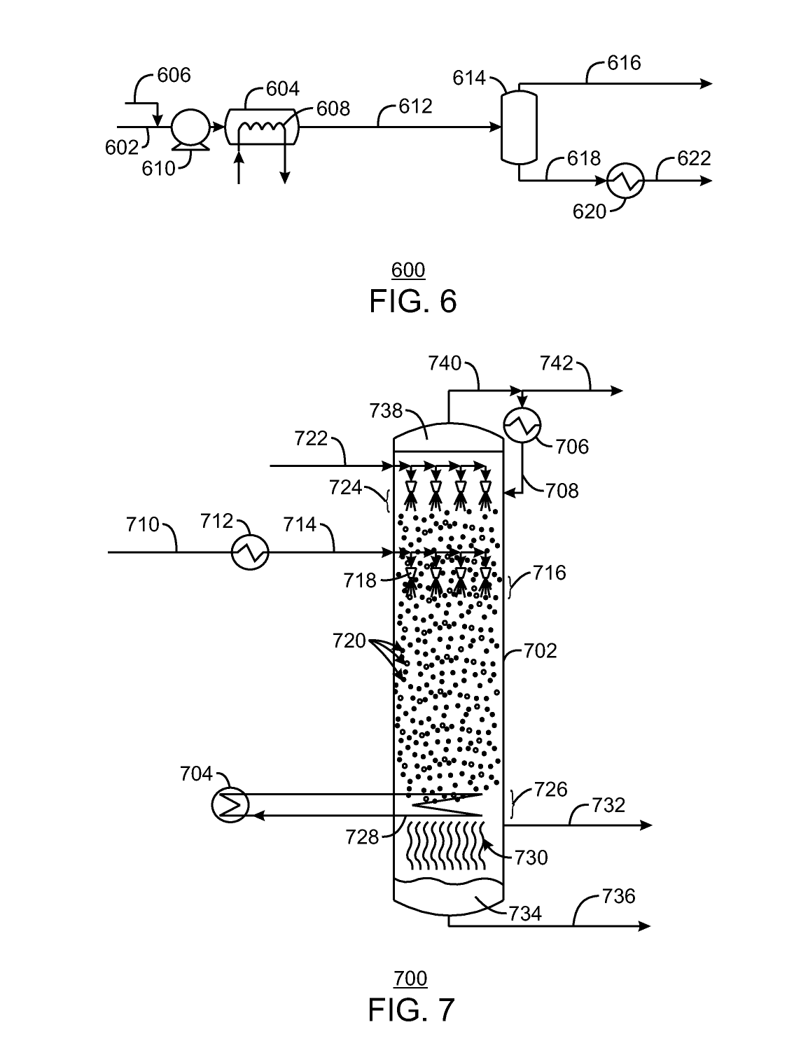

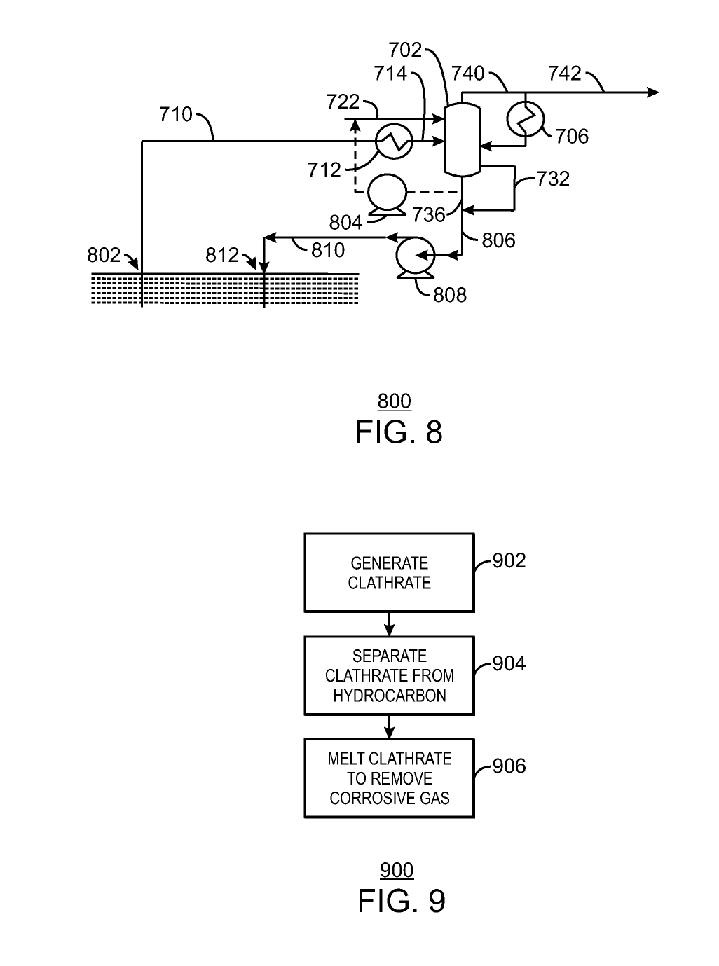

In an exemplary embodiment of the present techniques, the hydrocarbon (for example, a sour gas) is converted to a hydrate slurry and pumped through a pipeline to a final destination, as discussed with respect to FIG. 6, below. Removal of the corrosive components as far upstream in the process as possible may mitigate corrosion in elements of the transportation infrastructure, lessening the need for other prevention methods that may be more expensive or difficult to implement. Further, the corrosion inhibition effect is usefully coupled with flowable hydrate slurry formation processes such as cold flow or anti-agglomerate use in the oil and gas production industry. The techniques are not limited to forming flowable hydrate slurries, but could also have broader application in the fields of CO₂ sequestration and gas separation by hydrate formation, as discussed with respect to FIGS. 7 and 8, below.

### Systems for Generating Hydrates to Decrease Corrosion

FIG. 6 is a process flow diagram 600 illustrating a system for shipping a hydrate slurry in a hydrocarbon, in accordance with an exemplary embodiment of the present techniques. As shown in the process flow diagram 600, a raw hydrocarbon stream 602 containing corrosive gases is introduced to a hydrate reactor 604. Depending on the amount of water present in the raw hydrocarbon stream 602, an injector 606 may be used to add water to stoichiometrically balance the formation reaction, which may increase the amount of corrosive gases incorporated into the hydrate. If water injection is not desirable, other host molecules may be injected to form other types of clathrates. For example, hydroquinone may be injected to form a clathrate compound with H₂S. One of ordinary skill in the art will recognize that other compounds may also be selected as host compounds for forming clathrates with the corrosive gases. Further, anti-agglomerates may be added at the injector to lower the probability of hydrate agglomeration and increase the formation of a slurry.

The reactor 604 may be an in-line or static mixer or may be a continuous stirred tank reactor (CSTR). A heat exchanger 608 can be incorporated into the reactor 604 if the temperature is too high for hydrate formation, as determined from the equilibrium curves, discussed with respect to FIG. 2. Further, the heat exchanger may be used to add heat to raise the temperature above the equilibrium temperature for the formation of a methane hydrate with the hydrocarbon stream 602. If the pressure of the hydrocarbon stream is too low, a compressor 610 can be positioned upstream of the reactor 604 to increase the pressure.

After the hydrate is formed, a slurry 612 of the hydrate in the hydrocarbon can be shipped to a destination. In an exemplary embodiment, the reactor 604 is placed on the ocean floor, and the destination is the surface of the ocean. In other embodiments, the reactor 604 may be placed in a well, for example, near a natural gas reservoir, and used to form a hydrate slurry 602 in the hydrocarbon returning to the surface of the earth. As discussed herein, the hydrate slurry 610 may be less corrosive than the raw hydrocarbon stream 602.

At the destination, the hydrate slurry 612 can be used as a feed to a hydrate separator 614. The hydrate separator 614 divides the hydrate slurry 612 into a sweet stream 616 (containing substantially less corrosive gases than the raw hydrocarbon stream 602) and a hydrate stream 618. The hydrate separator 614 may be a conveyor belt or other physical separation device, or may be a version of the separation column discussed with respect to FIG. 7, below. The hydrate stream 618 can be sent to a melter, such as heater 620, which forms a corrosive gas/water stream 622 that may be further processed to remove the corrosive gas and isolate or purify the water or other host compound. In an exemplary embodiment, the separation stages discussed above are performed in a single separation column.

FIG. 7 is a process flow diagram 700 of a separation tower 702 that can use clathrates, such as hydrates, to separate corrosive gases from hydrocarbons, in accordance with an exemplary embodiment of the present techniques. The corrosive gases may be a single gas, such as CO₂, or may be a mixture of gases, including such gases as CO₂, H₂S, and others. As shown, the separation tower 702 does not use trays, packing, or other physical devices to entrain fluids or hydrates at certain levels, so the entire separation tower 702 is operated at a single pressure. However, the separation tower 702 is not limited to functioning without trays or packing, and other embodiments that use the formation of hydrates for separation may be designed with such devices. Even without physical trays, a number of equilibrium stages (i.e., theoretical trays) may be present at different levels, corresponding to different temperature points, in the separation tower 702. It may be useful to perform the separation using more than one equilibrium stage to achieve a desired gas purity.

In the separation tower 702, a hydrate equilibrium gradient can be imposed by heating the bottom of the tower to slightly above the equilibrium temperature (for example, 2° F., 5° F., or more) for dissolution of the hydrate of the corrosive gas impurity (e.g., CO₂, H₂S, or a mixture) using a heat exchanger 704. A reflux cooler 706 may be used to inject a cooled reflux stream 708 near the top of the tower. The reflux stream 708 can be cooled to slightly below the equilibrium temperature (for example, 2° F., 5° F., or more) for the hydrates of the corrosive gas mixture. The hydrocarbon feed 710, containing corrosive gases, can be cooled using a precooler 712 to a temperature close to the equilibrium temperature. The cooled feed 714 may then be injected into a feed zone 716 in the tower 702 at which the temperature is around the incipient (or formation) temperature for hydrates.

The cooled feed 714 can be passed through spray nozzles 718 that disperse the cooled feed 714 into a fine spray 720 in order to maximize the surface area of the water droplets, which may increase hydrate formation. A stream of water 722 can be injected into a conversion zone 724 in the tower 702 to react with corrosive gases rising from the feed zone 716.

In a melt zone 726 at the bottom of the tower 702, a flow from the heat exchanger 704 can be passed through a heating coil 728, which may dissociate the hydrate into a purified corrosive gas mixture 730. The purified corrosive gas mixture 730 may be removed from the tower 702 in a gas exit stream 732 at the location of the heating coil 728. Water 734, formed from the dissociation of the hydrate may be removed in a bottoms stream 736.

The sweetened hydrocarbon 738 can be removed as an exit stream 740. A portion of the exit stream 740 can be passed through the reflux cooler 706, and reinjected into the tower 702 as the cooled reflux stream 708. Another portion of the exit stream 740 can be removed as the sweetened hydrocarbon product 742.

In this exemplary embodiment, the separation tower 702 is oriented so that the sweetened hydrocarbon is removed from the top, and the water and corrosive gases are removed at the bottom. This configuration may be suitable for lighter hydrocarbons, such as natural gas. In other embodiments, for example, for liquid hydrocarbons, the separation tower 702 may be configured to have the sweetened hydrocarbon exit at the bottom of the separation tower 702 and the corrosive gases exit at the top of the separation tower 702. In an exemplary embodiment, the separation tower 702 is used to purify a hydrocarbon stream at a reservoir, allowing the sour water and corrosive gases to be reinjected into the reservoir to maintain reservoir pressure, as discussed with respect to FIG. 8.

FIG. 8 is a schematic 800 illustrating the use of the separation tower 702 to purify a hydrocarbon stream 710, in accordance with an exemplary embodiment of the present techniques. The reference numbers associated with the tower 702 are the same as discussed with respect to FIG. 7. In the schematic 800, a first well 802 can be used to produce the hydrocarbon feed 710 containing corrosive gases and sour water. The hydrocarbon feed 710 can be processed in the tower 702 to remove the corrosive gases and sour water, generating a sweetened hydrocarbon 742. To conserve water, the bottoms stream 736 from the tower 702 may be circulated by a pump 804 to be used as a source of water for the stream of water 722 injected into the conversion zone of the tower 702. The remainder of the bottoms stream 736 may be combined with the corrosive gas 732 to form an injection stream 806.

The injection stream 806 may be pressurized by a pump 808 to form a pressurized injection stream 810. The pressurized injection stream 810 can then be injected into a formation through a second well 812. The injection may be placed into the active reservoir from which the hydrocarbon is being produced, or into a different formation, such as an empty (produced) hydrocarbon reservoir. If the injection takes place into the active reservoir, it may help to maintain the formation pressure and, thus, production rates.

The system is not limited to that shown in the schematic 800. For example, if a reactor is placed downhole to prevent corrosion in the well casing and production lines, the raw hydrocarbon feed 710 may already be a slurry when it is injected into the separation tower 702. In this case, the separation tower 702 may be used as a separator and melter. The reconfiguration may be performed, for example, by decreasing or eliminating the water feed 722 to the tower 702.

In other embodiments, the configuration discussed with respect to FIG. 6 may be used for processing a hydrocarbon at a field. In this case, a downhole reactor may be used to form a slurry that is produced at the surface and injected into a separator 614 (FIG. 6). The separator 614 can remove the hydrate from the slurry, producing a sweet stream 616. The hydrate may be sent to a melter, for example, heater 620, prior to being compressed by a pump, such as pump 808 in FIG. 8) and injected into a formation. One of ordinary skill in the art will recognize that any number of other configurations may be useful for separating out corrosive gases by the formation of hydrates or other clathrates.

FIG. 9 is a block flow chart of a method for using clathrates to remove corrosive gases from hydrocarbons, in accordance with exemplary embodiments of the present techniques. The method 900 begins at block 902 with the generation of a clathrate of a corrosive gas mixture, including gases such as CO₂ and H₂S, among others. The clathrate can be a clathrate hydrate, or hydrate, as discussed herein. In other embodiments, the clathrate can be formed from other host molecules, such as hydroquinone, among others. The clathrate may be generated in a reactor, which can include in-line mixers, among others. Heat may be added to or removed from the hydrocarbon to control the temperature of the formation. In other embodiments, the clathrate can be generated in a single tower that also functions as a separator and melter.

At block 904, the clathrate is separated from the hydrocarbon, for example, using a physical device such as a conveyor belt or spinning drum separator. In other embodiments, the clathrate may be separated by falling through a tower such as discussed with respect to FIG. 7, which also functions as a reactor and melter.

At block 906, the clathrate is melted or otherwise dissociated to remove the corrosive gas mixture from the host molecule. In the case of a hydrate, this procedure forms a corrosive gas mixture and sour water, i.e., water that is contaminated by some residual amount of the corrosive gases. The corrosive gas mixture and sour water may be injected into a well to sequester the corrosive gases.

### EXAMPLES

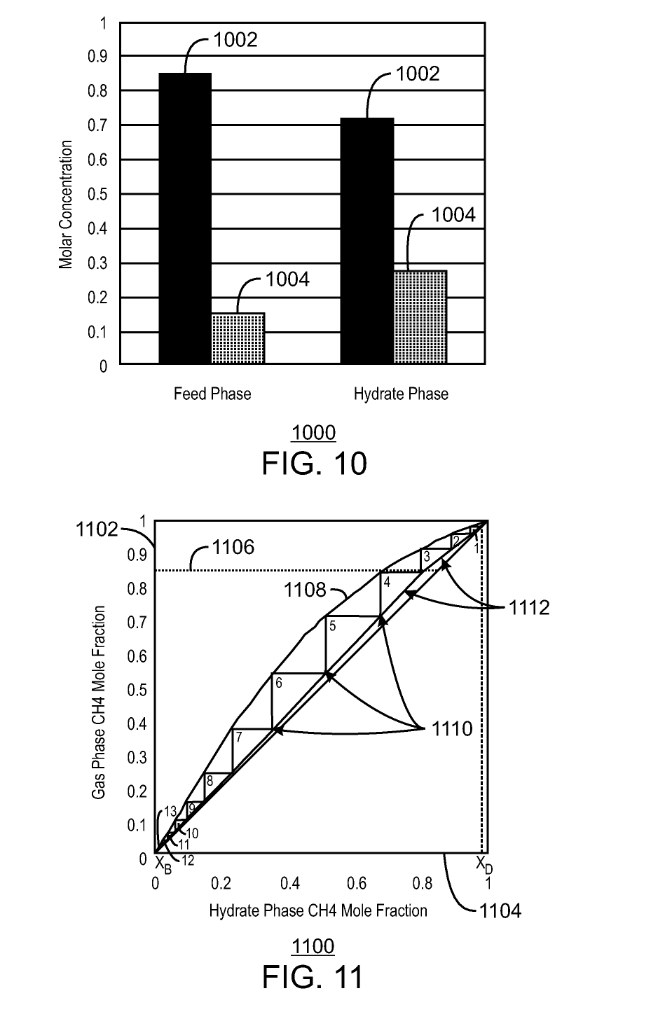

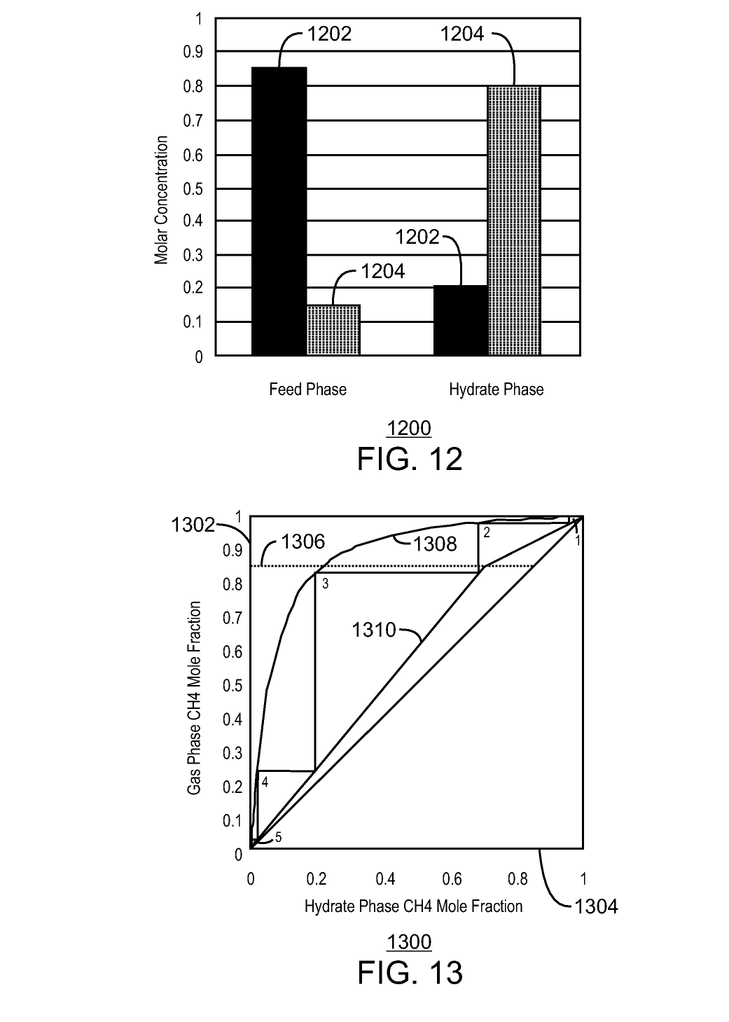

Example calculations were performed to determine the theoretical efficiency of using hydrates to separate mixtures of 85 mol % CH₄ with either 15 mol % CO₂ or H₂S. The separation of a mixture of 85 mol % CH₄ and 15 mol % CO₂ is discussed with respect to FIGS. 9 and 10. The separation of a mixture of 85 mol % CH₄ and 15 mol % H₂S is discussed with respect to FIGS. 11 and 12.

FIG. 10 is a bar chart 1000 comparing the mole fractions of CH₄ 1002 and CO₂ 1004 in a feed phase and a hydrate phase, in accordance with exemplary embodiments of the present techniques. The calculations were performed at 38° F. and the incipient pressure of 440 psia. The data is summarized in Table 1, below. As shown in FIG. 10 and Table 1, CO₂ is twice as concentrated in the hydrates than in the original gas composition and, thus, the CO₂ may be separated from methane using hydrates.

TABLE 1
Comparison of molar fraction

| | | | CO2:CH₄ |
|---|---|---|---|
| | mole fraction CH₄ | mole fraction CO2 | moles/moles |
| Feed Phase | 0.85 | 0.15 | 0.176 |
| Hydrate Phase | 0.72 | 0.28 | 0.385 |

The separation of the methane from the CO₂, may be performed in a column or tower, as discussed with respect to FIG. 7, above. Although the equilibria discussed with respect to FIG. 2 are not based on vapor pressure differences, the difference between the equilibrium concentrations in each of the phases indicates that a McCabe-Thiele plot can be used to design a separation column for purification of the methane, such as the tower 702 discussed with respect to FIG. 7.

FIG. 11 is a McCabe-Thiele plot 1100 for a theoretically staged separation column (or tower) for the separation of CO₂ from methane, in accordance with an exemplary embodiment of the present techniques. In the McCabe-Thiele plot 1100, the y-axis 1102 represents the mole fraction of CH₄ in the gas phase, while the x-axis 1104 represents the concentration of CH₄ in the hydrate phase. It should be understood that the values shown along each of the axes 1102 and 1104 can be subtracted from one to determine the concentration of CO₂ in the respective phase. The mole fractions of the feed to the column 1106 are the same as discussed above, 15 mol % CO₂ and 85 mol % methane, and the pressure is at 200 psig. The temperatures for an input precooler, a reflux cooler, and a heater can be selected as discussed with respect to FIG. 7.

The McCabe-Thiele plot 1100 shows the number of theoretical stages (numbered one to 13 in this example), which can be used to determine column height. The theoretical stages are bound within the equilibrium curve 1108 and the points of concentration parity 1110 (i.e., points at which the concentration of a component leaving one stage is equal to the concentration of the same component entering the next stage). Operating lines 1112 are drawn along the points of concentration parity 1110. FIG. 11 shows that the separation of the CO₂/CH₄ mixture into two exit streams, one of 98 mol % pure CH₄ and one of 98 mol % pure CO₂, would require 13 theoretical stages using a reflux of 3.

FIG. 12 is a bar chart 1200 comparing the mole fractions of CH₄ 1202 and H₂S 1204 in a feed phase and a hydrate phase, in accordance with exemplary embodiments of the present techniques. The calculations were performed at 38° F. and the incipient pressure of 108 psia. The data is summarized in Table 2. As shown in FIG. 11 and Table 2, H₂S is 22 times as concentrated in the hydrates than in the original gas composition and, thus, H₂S can be separated from methane using hydrates.

FIG. 13 is a McCabe-Thiele plot 1300 for a theoretically staged separation column for H₂S separation from methane, in accordance with an exemplary embodiment of the present techniques. Similar to the McCabe-Thiele plot 1100 discussed with respect to FIG. 11, the y-axis 1302 represents the mole fraction of CH₄ in the gas phase, while the x-axis 1304 represents the concentration of CH₄ in the hydrate phase.

TABLE 2
Comparison of molar fraction

| | | | H₂S:CH₄ |
|---|---|---|---|
| | mole fraction CH₄ | mole fraction H₂S | moles/moles |
| Feed Phase | 0.85 | 0.15 | 0.176 |
| Hydrate Phase | 0.20 | 0.80 | 3.938 |

It should be understood that the values shown along each of the axes in the McCabe-Thiele plot 1300 may be subtracted from one to determine the concentration of H₂S in the respective phase. The mole fractions of the feed to the column 1306 are the same as discussed above, 15 mol % H₂S and 85 mol % CH₄, and the pressure is 200 psig. The temperatures for the input precooler, reflux cooler, and heater can be selected as discussed with respect to FIG. 7. FIG. 12 shows that H₂S and CH₄ has a much wider operating window (i.e., the separation between the equilibrium curve 1308 and the operating lines 1310) than for the separation of CO₂ from CH₄, leading to more efficient separation. Separation of the H₂S/CH₄ feed gas into >99.9% pure CH₄ and >99.9% pure H₂S requires five theoretical stages (number one through five in FIG. 13) at a reflux ratio of one.

While the present techniques may be susceptible to various modifications and alternative forms, the exemplary embodiments discussed above have been shown only by way of example. However, it should again be understood that the techniques is not intended to be limited to the particular embodiments disclosed herein. Indeed, the present techniques include all alternatives, modifications, and equivalents falling within the true spirit and scope of the appended claims.

What is claimed is:

1. A method for isolating a corrosive gas in a hydrocarbon stream, comprising:
reacting a host compound with the hydrocarbon stream comprising the corrosive gas, wherein a pressure of the reaction, a temperature of the reaction, or both, are controlled to maximize formation of a clathrate of the corrosive gas and minimize formation of a clathrate of a hydrocarbon in the hydrocarbon stream;
separating the clathrate of the corrosive gas from the hydrocarbon stream; and
melting the clathrate of the corrosive gas to remove the corrosive gas.

2. The method of claim 1, further comprising:
placing a reactor configured to form the clathrate of the corrosive gas at a first location in a hydrocarbon transport system;
placing a separator configured to remove the clathrate of the corrosive gas at a second location in the hydrocarbon transport system; and
placing a melter configured to melt the clathrate of the corrosive gas at a third location in the hydrocarbon transport system.

3. The method of claim 1, further comprising:
reacting the host compound with the hydrocarbon stream at a wellhead to form the clathrate of the corrosive gas; and
pumping a slurry comprising the hydrocarbon and the clathrate of the corrosive gas to a destination.

4. The method of claim 1, further comprising:
forming a slurry of the clathrate of the corrosive gas in a reservoir; and
flowing the slurry to a separation system at a surface.

5. The method of claim 1, further comprising reinjecting a produced sour water, the corrosive gas, or both, into a producing reservoir.

6. The method of claim 1, further comprising injecting a produced sour water, the corrosive gas, or both, into a non-producing reservoir.

7. The method of claim 1, further comprising adding an anti-agglomerate to the hydrocarbon stream.

8. The method of claim 1, further comprising adding of a corrosion inhibitor to the hydrocarbon stream, using a cathodic protection system, applying a coating to a metal surface in a hydrocarbon transport system, forming a part in the hydrocarbon transport system from a corrosion resistant alloy, or any combinations thereof.

9. A system for transporting a hydrocarbon through a transportation infrastructure, comprising:
a reactor configured to form a clathrate between a host compound and a corrosive gas in a hydrocarbon stream, wherein the reactor comprises a heat exchanger configured to control a temperature of the reactor to minimize a formation of a hydrocarbon clathrate;
a separator configured to remove the clathrate from the hydrocarbon stream; and
a melter configured to melt the clathrate and release the corrosive gas.

10. The system of claim 9, further comprising a pipeline configured to transport a slurry comprising the clathrate in the hydrocarbon stream.

11. The system of claim 9, further comprising a vessel configured to function as the reactor, the separator, and the melter.

12. The system of claim 9, further comprising a vessel configured to function as the separator and the melter.

13. The system of claim 9, wherein the reactor comprises a static mixer.

14. The system of claim 13, further comprising a water injection port upstream of the static mixer.

15. The system of claim 9, further comprising an injection system configured to inject the corrosive gas released from melting the clathrate into a well.

16. The system of claim 9, wherein the host compound comprises water.

17. The system of claim 9, wherein the corrosive gas comprises carbon dioxide, hydrogen sulfide, or any combination thereof.

18. A method for producing a hydrocarbon, comprising:
producing a hydrocarbon stream, wherein the hydrocarbon stream comprises a corrosive gas;
reacting a host compound with the hydrocarbon stream to form a slurry of a clathrate of the corrosive gas in the hydrocarbon stream; and
transporting the slurry to a destination through a pipeline.

19. The method of claim 18, further comprising:
separating the clathrate from the hydrocarbon stream; and
melting the clathrate to remove the corrosive gas.

20. The method of claim 18, further comprising:
melting the clathrate in the pipeline; and
separating the corrosive gas from the hydrocarbon stream at the destination.
